(19) United States
(12) Patent Application Publication
(10) Pub. No.: US 2015/0273119 A1
(43) Pub. Date: Oct. 1, 2015

(54) FORMULATION COMPRISING ANTI-SCARRING AGENTS AND BIOCOMPATIBLE POLYMERS FOR MEDICAL DEVICE COATING

(71) Applicant: SNU R&DB FOUNDATION, Seoul (KR)

(72) Inventors: Chan Yeong HEO, Gyeonggi-do (KR); Young Bin CHOY, Gyeonggi-do (KR); Sung Yoon CHOI, Seoul (KR); Byung Hwi KIM, Gyeonggi-do (KR); Beom Kang HUH, Seoul (KR); Min PARK, Seoul (KR)

(73) Assignee: SNU R&DB FOUNDATION, Seoul (KR)

(21) Appl. No.: 14/669,777

(22) Filed: Mar. 26, 2015

Related U.S. Application Data
(60) Provisional application No. 61/970,452, filed on Mar. 26, 2014.

(51) Int. Cl. A61L 31/16; A61L 27/54; A61L 27/34; A61L 31/10

(52) U.S. Cl. CPC A61L 31/16; A61L 31/10; A61L 27/54; A61L 27/34; A61L 2420/06

(57) ABSTRACT

Disclosed is a formulation for coating a medical device which comprises an anti-scarring agent and a biocompatible polymer for regulating the release of the anti-scarring agent, wherein the mass ratio of the biocompatible polymer to the anti-scaring agent is from 100:15 to 100:0 in which 0 is not included. The present formulation can be advantageously used for coating medical devices for a sustained release of the agents from the device, thus effectively minimizing or preventing the formation of scar tissues resulted from the use of implantable and non-implantable medical devices.

### CROSS-REFERENCE TO RELATED APPLICATIONS

The present application claims the benefit of U.S. Provisional Application No. 61/970,452 filed Mar. 26, 2014, the disclosure of which is incorporated herein.

### BACKGROUND OF INVENTION

1. Field of the Invention

The present disclosure generally relates formulations for coating medical devices for a sustained drug release.

2. Description of the Related Art

Medical devices are widely used in the medical field for early detection, prevention and treatment of disorders. For example medical textiles such as sutures and wound dressings are used to heal surgical wounds after surgery. In particular, functional textiles which can control the side effects after surgery are limited to suppress infections or inflammations. Currently, however, problems on the rise in addition to the common side effects such as infections and pain are the formation of scar tissue during the healing process which resulted from the abnormal regeneration of tissue. However there are no fundamental solutions for the problems.

Korean Patent No. 0439156 relates to compositions for coating drug-eluting stent and methods for preparing the same and discloses agents formed by coprecipitation of biologically active materials and water-soluble polymer and compositions comprising a cross-linked polymer solution.

Korean Patent No. 1248368 relates to sutures comprising polymer film loaded with medicine and method for preparing the same and discloses sutures the surface of which are wrapped with the sheets comprising a biodegradable polymer layer.

US Patent Application No. 2011/0264139 relates to compositions for coating substrate comprising hydrogen radicals and discloses compositions for coating which comprises hydrophilic polymer mixture of at least two different molecular weights and has functional groups which can be cross-linked by UV.

The above documents do not disclose at all regarding formulations for coating for suppressing scar tissue formation.

Particularly there are needs to develop coating compositions or formulations for coating implantable or non-implantable medical devices for a sustained drug release from the device over a certain period of time sufficient to effectively suppress the formation of scar tissue.

### SUMMARY OF THE INVENTION

The present disclosure is to provide compositions or formulations for coating a medical device comprising anti-scarring agents and biocompatible polymer.

In one embodiment, the biocompatible polymer and anti-scarring agent for regulating the release of the anti-scarring agent is included in the ratio of about 100:15 to 0, in which 0 is not included. In other embodiment the ratio is about 100:15 to about 100:1.

The implantable devices used for a medical purpose and includes but is not limited to wound dressings, products for wound closure, disposable bands, medical sponges, structures for treating urinary incontinence, structures for fixing organs, meshes for preventing stenosis, maxillofacial meshes, hernia meshes, fibrous structures for heart valves, fibrous structures for cartilage regeneration, stents, stent-grafts, catheter, guidewires, coils for nerve blood vessel unruptured intracranial aneurysms, balloon, filter (for example, white blood cell purification filter, blood purification filter, filter for intravenous injection, filter for blood transfusion, filter for dialysis and filter for heart lung machine), dental textiles, vascular graft, intraluminal paving system, pacemaker, electrodes, leads, defibrillator, joint and bone implants, spinal implants, silicone implants, access port, intra aortic balloon pumps, heart valves, sutures, artificial hearts, artificial blood vessels, artificial ligament, artificial kidney, artificial cochlea, artificial corneas and other medical devices which needs to be coated for anti-scarring function.

In one embodiment, using the present formulation the drug contained therein is controlled released by a least two properties of the polymer including hydrophobicity, molecular weight, network structure and degradation rate.

The foregoing summary is illustrative only and is not intended to be in any way limiting. Additional aspects and/or advantages of the invention will be set forth in part in the description which follows and, in part, will be obvious from the description, or may be learned by practice of the invention.

### ADVANTAGEOUS EFFECTS

The present formulation comprising an anti-scarring agent and a biocompatible polymer can be advantageously used for coating medical devices for a sustained release of the agents from the device, thus effectively minimizing or preventing the formation of scar tissues resulted from the use of implantable and non-implantable medical devices.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a graph showing the cumulative amount of drug released over a period of time from the sutures coated with the formulation according to one exemplary embodiment of the present disclosure.

FIG. 2 is a graph showing the cumulative amount of drug released over a period of time from the prostheses coated with the formulation according to one exemplary embodiment of the present disclosure.

FIG. 3A is an image of the incision (indicated by arrows) on the back of a mouse at 5 days after the incision was sutured with the sutures (SDS and MDS) coated with the formulation according to one exemplary embodiment of the present disclosure.

FIG. 3B is an image of the incision on the back of a mouse at 21 days after the incision was sutured with the sutures (SDS and MDS) coated with the formulation according to one exemplary embodiment of the present disclosure.

FIGS. 4A, 4B and 4C are microscopic images showing the directions of collagen formed at the wound site, in which the samples were biopsied at 5, 12 and 21 days, respectively after suturing the wounds as in FIGS. 3A and 3B (X200, scale bar: 100 μm).

FIG. 5 is an image showing the procedure for implanting the prosthesis coated with the formulation according to one exemplary embodiment of the present disclosure to the back of a mouse.

FIG. 6A is a graph showing a thickness of the scar tissue at 1, 2, 4, 8 and 12 weeks after implanting the prosthesis as in FIG. 5.

FIG. 6B is a graph showing density of the collagen at 1, 2, 4, 8 and 12 weeks after implanting the prosthesis as in FIG. 5.

FIGS. 7A to 7E are microscopic images of H&E stained tissues biopsied from the implanted site at 1, 2, 4, 8 and 12 weeks, respectively after implanting the prosthesis as in FIG. 5, showing thickness of the scar tissue (X50, scale bar: 1 mm).

FIGS. 8A to 8E are microscopic images of the tissues biopsied from the implanted site at 1, 2, 4, 8 and 12 weeks, respectively after implanting the prosthesis as in FIG. 5 and stained with Masson's trichrome, showing thickness of the scar tissue (X200, scale bar: 100 μm).

FIG. 9 is a microscopic image showing the direction of collagens formed during the normal wound healing process (left) and the hypertrophic scarring (right) process in which the former shows the accumulation of collagen fibers having a regular directionality compared to the latter showing the irregular directionality of the collagen fibers.

In the figures above, IM indicates a group received silicon prostheses; PLGA_IM: silicon prosthesis coated formulation comprising only the polymer; TR_IM: silicon prosthesis coated formulation comprising only the anti-scarring agent; PLGA_TR_IM: silicon prosthesis coated formulation comprising the polymer and the anti-scarring agent in a mass ratio of 100:1.

### DETAILED DESCRIPTION OF THE EMBODIMENTS

In one aspect, the present disclosure relates to a formulation for coating a medical device comprising an anti-scarring agent and a biocompatible polymer for regulating the release of the anti-scarring agent.

The term “medical device” as used herein refers to any implantable or non-implantable devices used for a medical purpose and includes but is not limited to wound closure, disposable bands, medical sponges, structures for treating urinary incontinence, structures for fixing organs, meshes for preventing stenosis, maxillofacial meshes, hernia meshes, fibrous structures for heart valves, fibrous structures for cartilage regeneration, stents, stent-grafts, catheter, guidewires, coils for nerve blood vessel unruptured intracranial aneurysms, balloon, filter (for example, white blood cell purification filter, blood purification filter, filter for intravenous injection, filter for blood transfusion, filter for dialysis and filter for heart lung machine), dental textiles, vascular graft, intraluminal paving system, pacemaker, electrodes, leads, defibrillator, joint and bone implants, spinal implants, silicone implants, access port, intra aortic balloon pumps, heart valves, sutures, artificial hearts, artificial blood vessels, artificial ligament, artificial kidney, artificial cochlea, artificial corneas and other medical devices which needs to be coated for anti-scarring function.

The term “anti-scarring agent or drug”, “agent or drug for suppressing the formation of scar tissue”, “anti-fibrosing agent or drug”, and “agent or drug for suppressing fibrosis”, as used herein are used interchangeably and refer to a medication that prevents or suppresses the formation of scar tissue through mechanisms that suppresses inflammations or acute inflammatory responses, the generation or activation of cytokines, the migration or proliferation of cells for connective tissues (for example fibroblasts, smooth muscle cells, vascular smooth muscle cells and the like), vasculogenesis, reconstruction of tissues and/or that reduce the generation of extra cellular matrix or accelerate the degradation of extra cellular matrix.

The anti-scarring agents which may be used for the present disclosure include angiogenesis inhibitors, agonists or antagonists for 5-lipoxygenases, chemokine receptor agonists CCR (1, 3 and 5), cell cycle inhibitors, cyclin dependent protein kinase inhibitors, EGFs (Epidermal Growth Factor), receptor kinase inhibitors, elastase inhibitors, Factor Xa inhibitors, farnesyl transferase inhibitors, fibrinogen agonists, guanylate cyclase activators, IL-4 agonists and immunomodulators, for example, acetmetacin, acrivastine, aldosterone, antazoline, astemizole, azatadine, azelastine, beclometasone, betamethasone, bromfenac, buclizine, carprofen, cetirizine, chloropyriline, chloropheniramine, clemastine, cromolyn, cyclizine, cyproheptadine, dexamethasone, diazoline, diclofenac, diphenhydramine, ebastine, emedastine, epinastine, etodolac, fenbufen, fenoprofen, fexofenadine, fludrocortisone, flurbiprofen, flurometalone, hydroxyzine, ibuprofen, indometacin, ketoprofen, ketorolac tromethamine, ketotifen, levocabastine, levoceterizine, lodoxamide, loratadine, loteprednol, loxoprofen, medrysone, mepivacaine, mequitazine, methdilazine, methapyrilene, monteleukast, nabumetone, naphazoline, naproxen, nedocromil, norastemizole, norebastine, olopatadine, phenidamine, phenylephrine, oxatamide, oxymetazoline, pemirolast, pheniramine, picumast, prednisilone, promethazine, rimexalone, repirinast, sulindac, suprofen, zafirlukast, tetrahydozoline, terfenadine, tiaprofenic acid, tometim, tranilast, triamcinolone, trimeprazine and triprolidine, or pharmaceutically acceptable salts thereof but are not limited thereto.

In one embodiment, the present formulation comprises an anti-scarring agent that suppresses the generation or activity of cytokines at the site of wound. For example, those suppress the activity of TGF-β, which plays a major role in the formation of scar tissue for example tranilast or a pharmaceutically acceptable salts thereof are included.

TGF-β is a key cytokine that initiates and terminates a repair of the damaged tissues and thus its continued expression results in the fibrosis of tissues. Therefore, the suppression of TGF-β is able to inhibit the formation of scar tissue by preventing excessive cellular activities and proliferations.

Also included in the present formulation is at least one release control material which is a biocompatible and/or biodegradable polymer.

The term “release control material” as used herein refers to a biocompatible and/or biodegradable polymer to which the anti-scarring agents are loaded and which is able to control or regulate the amount or the period of time of the drug released. That is achieved by regulating or controlling characteristics or properties of the polymer including such as hydrophilicity/hydrophobicity, porosity, molecular weight, structure of the networks, surface charges, degradation rates and the like

The term “biocompatible” as used herein refers to a property of a material that does not cause substantially harmful response to the subject when introduced to a subject. For example, it means that when materials or devices which are foreign to a subject are used, they do not induce substantially harmful reactions such as inflammatory reaction and/or immune reactions. Biocompatible materials which may be used for the present disclosure include biodegradable or biosafety materials.

The term “biodegradable polymer” as used herein refers to a polymer which is degraded into a low molecular weight material through a degradation process such as a hydrolytic reaction or enzymatic reaction during the metabolism of an organism. In one embodiment, the biocompatible polymer is polylactide (PLA), polyglycolide (PGA), poly(lactic-co-glycolic acid) (PLGA), polyorthoester, polyanhydride, polyamino acid, polyhydroxybutyric acid, polycaprolactone, polyalkylcarbonate, ethyl cellulose, chitosan, starch, guargum, gelatin, or collagen, but is not limited thereto.

In the present disclosure, to delay or control the time the drug is released from the formulation, the amount of polymers employed is increased such that the drug continues to be released over a period of time in a controlled manner from a formulation. In the present formulation, the ratio of the biocompatible polymer to the anti-scarring agents is from about 100:15 to 100:0 by weight in which zero is not included. In one embodiment, the ratio of the biocompatible polymer to the anti-scarring agents is about 100 to 15 to 100 to 1 but the ratio below 100:1 is not excluded.

By employing the biocompatible polymers and anti-scarring agents in a ratio as described above in the present formulation, the anti-scarring agent comprised in the formulation is released over a period of time of at least 3 days, and thus the formation of scar tissues is prevented more effectively in comparison to the cases where the anti-scarring agent is released not in a controlled manner and thus the wound site is exposed to the drug only for a short period time, for example, one day. In one embodiment, when the ratio of the biocompatible polymer to the anti-scarring agents is from about 100:15, the drug is released for duration of at least 3 days. In other embodiment, when the ratio of the biocompatible polymer to the anti-scarring agents is from about 100:1, the agent is released over a period of at least 14 days.

In a further embodiment, as a biocompatible polymer, PLAG is used, and as an anti-scarring agent, tranilast is used, but the polymer and drug are not limited thereto. By using PLGA, particularly due to its suitable hydrophobicity, molecular weight, structure of the network and degradation rate in the present formulation, the drug is able to be released in a controlled manner. Within the ratio of PLGA to the anti-scarring agent as disclosed herein, as the ratio of PLGA to anti-scarring agent is increased, the duration of the drug release is also increased due to the increased action of the polymer. It is preferable that the polymer is not degraded until the drug release is completed. Also, the polymer may be degraded with the implanted medical device when the device is made of biodegradable material.

The present formulation is used for coating the medical devices as stated above, and thus may be prepared in the form of a sheet, a powder, a layer, a solution or a composition. In one embodiment, the present formulation is prepared in the form of a composition. In other embodiment, the present formulation is prepared in the form of a sheet or film, and used for coating sutures which is then used for suturing wounds. The term “wounds” as used herein refers to damage to the body part so that the structural intactness of the damaged body part is lost. In one perspective, the wounds include a surgical site. In other perspective, the wounds include a contused wound, a cutting wound, a lacerated wound, a nonpenetrating injury (that is, a lanceolate with a damage under the skin without an open wound), an open wound, a penetrating wound, a perforating lanceolate, a puncture wound, a septic wound, a subcutaneous wound and a burn and the like but are not limited thereto.

In other embodiment, the present formulation may be prepared in the form of a composition and used for coating prostheses such as breast prosthesis.

The term “coating” as used herein refers to attach the present composition to the medical device of interest. For example, the attachment includes an attachment through a surface adsorption, a dipping or immersion, a covalent or monovalent bonding, ionic bonding or a simple collision and the like but is not limited thereto. The methods for coating are known in the related art, which may be referred for example in Y, Ikada Biomaterials (1994) 15: 725-736; Antibacterial poly(D,Llactic acid) coating of medical implants using a biodegradable drug delivery technology; and Golwitzer et al., Journal of Antimicrobial Chemotherapy (2003) 51: 585-591. The present composition is coated on the medical device using an electrospinning or a solution casting method.

The present disclosure is further explained in more detail with reference to the following examples. These examples, however, should not be interpreted as limiting the scope of the present invention in any manner.

### EXAMPLES

### Example 1

### Preparation of Composition Coating Sutures

Poly (lactic-co-glycolic acid) (PLGA; 50:50; inherent viscosity=0.41 dl/g)(Lakeshore Biochemicals, USA) and tranilast (JW Pharmaceutical, Korea) as an anti-scarring agent was mixed at a ratio of 100:15 (denoted as SDS, single layer drug sheet) and 100:8.5 (denoted as MDS,) by weight.

Specifically for the SDS (single layer drug sheet), 1.5 g PLGA and 0.225 g tranilast were dissolved together in 5 ml of a mixture of organic solvent (Dichloromethane (DCM), Tetrahydrofuran (THF), dimethylformamide (DMF)=3:1:1 v/v/v) to prepare a polymer and drug solution. Then the polymer and drug solution was electrospun to prepare a film, which was then used for coating sutures. For the electrospinning, a 10 ml syringe was filled with the polymer and drug solution and connected to a 26G needle tip. A steel plate of 30 cm×7 cm was attached to a cylindrical collector for electrospinning. The spinning rate for the cylindrical collector was fixed at 100 rpm and the distance from the tip of the syringe was kept at 10 cm. A voltage of 15 kV was applied while a total of 1 ml polymer and drug solution was fed at 3 ml/h.

Specifically for the MDS (Multi-layered drug sheet), 1.5 g PLGA and 0.3825 g tranilast were dissolved together in a 5 ml of a mixture of organic solvent (Dichloromethane (DCM), Tetrahydrofuran (THF), dimethylformamide (DMF)=3:1:1 v/v/v) to prepare a polymer and drug solution. Only 1.5 g PLGA was dissolved in a 5 ml of a mixture of organic solvent (Dichloromethane (DCM), Tetrahydrofuran (THF), dimethylformamide (DMF)=3:1:1 v/v/v) to prepare a polymer solution. Then the resulting solutions were electrospun to be used for coating sutures. For the electrospinning, a 10 ml syringe was filled with a polymer and drug solution or a polymer solution and connected to a 26G needle tip. A steel plate of 30 cm×7 cm was attached to a cylindrical collector for electrospinning. The spinning rate for the collector was fixed at 100 rpm and the distance from the tip of the syringe was kept at 10 cm. A voltage of 15 kV was applied, where the solutions were fed at 3 ml/h in the order of a 0.5 ml of the polymer solution, a 1 ml of the polymer and drug solution, and a 0.5 ml of the polymer solution to prepare MDS.

For coating sutures, the SDS and MDS formulation prepared as above were cut into strands, which were then used to wrap sutures (VICRYL/W9114, Ethicon, USA) and treated at a glass transition temperature of 47° C. for 1 hour to attach the strand to the surface of sutures.

The drug released from the sutures treated as above was measured as below. For a quantitative measurement, the coated sutures were cut into 2 cm in length and dissolved in a 10 ml of organic solvent DMF (dimethyl formaldehyde). Then the absorbance of the solution was measured at 332 nm using a spectrophotometer (UV-1800, Shimadzu, Japan). Then concentration of the drug in the solution was determined by the following formula: Converted Concentration of the drug (mg/ml)=11.9×Optical Density. The formula was made from the optical densities obtained using DMF solutions containing various known amounts of the drug. Then the actual amount of drug loaded on a 2 cm of the sutures was determined from the converted concentration by multiplying 10 ml to the converted value, which was then divided by two to obtain the actual drug amount per cm of the sutures.

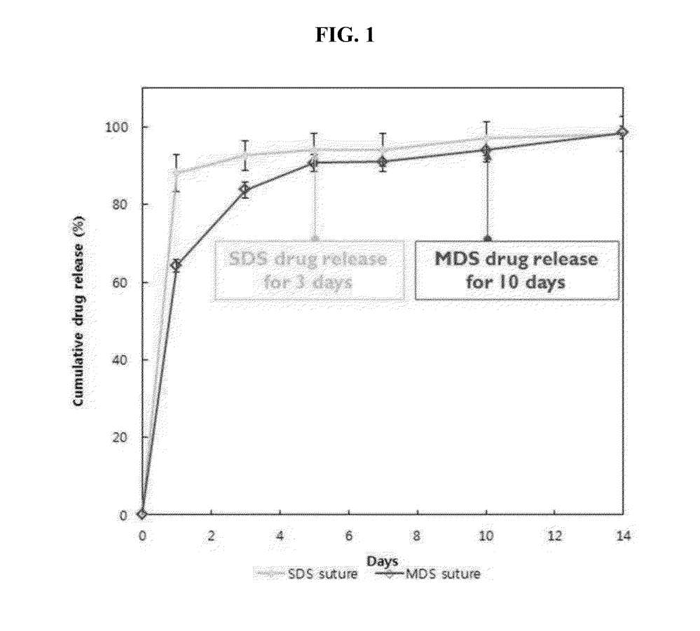

For in vitro profiles, 4 cm of the sutures in a test tube containing Phosphate buffered saline (PBS; pH 7.4) was incubated at 37° C. while shaking. Then at 1, 2, 3, 5, 7, 10, and 14 days after the incubation, 1 ml of the solution was obtained and 1 ml of fresh PBS was added back. The solution obtained then was subject to a spectrophotometric measurement using a spectrophotometer (UV-1800, Shimadzu, Japan) to obtain an optical density, from which the converted concentration was obtained by multiplying 8.9 to the O.D. value measured. The formula was made from the optical densities obtained using PBS solutions containing various known amounts of the drug. A graph of showing the cumulative amount of the drug released was calculated as above at the indicated dates as in FIG. 1.

FIG. 1 shows the cumulative amount of the drug released from the coated sutures, in which the drug was controlled released for 3 days for SDS and for 10 days for MDS. These results indicate that the higher the ratio of the polymer contained in the formulation relative to the amount of the drug is, the longer the period of the drug is released thus being more effective in suppressing the formation of scar tissue.

### Example 2

### Preparation of Formulation for Coating Prostheses and Coating Prostheses Using the Same

TR-IM was prepared as below.

First 50 mg of tranilast was dissolved in 50 ml of DMF, the solution was put into a container of the spraying machine self-manufactured and a nozzle hole for spraying was set to 0.8 mm and the pressure was set to 1.03 bar. Then silicone implant currently in clinical use (SFS-LP, Hans Biomed, Korea) was punched out at the size of 2 m in diameter and 1.5 mm in thickness, which was then placed on a Teflon mount of 1.5 mm in diameter. The distance from the sample to the nozzle was set to 20 cm. Then the sample was spray coated for 2 sec and dried for 30 min at room temperature. Then, the spray coating was repeated at the same condition for 2 sec followed by drying the sample under vacuum for one day to remove the residual organic solvent. Then with the coated side facing upward, epoxy was used to bond the coating to the prosthesis, which was then hardened at 37° C. for one day to obtain the prosthesis coated on both sides.

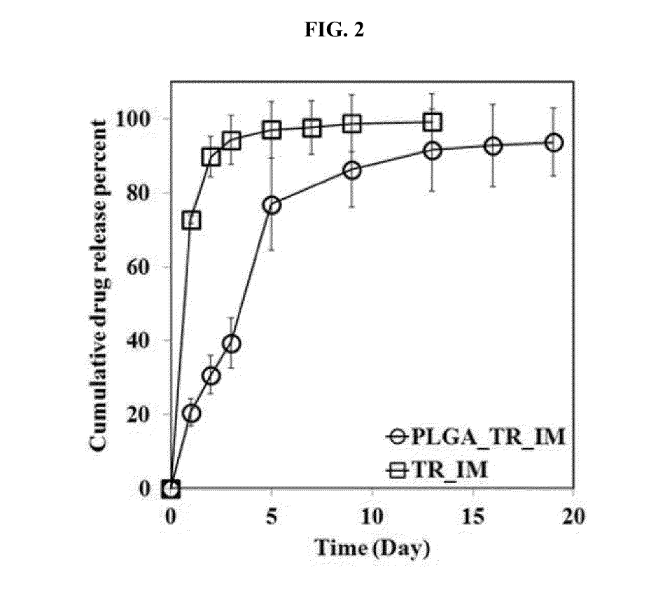

The coated prostheses were then immersed in 5 ml of PBS (pH 7.4, Tween 20 1% v/v) and incubated in a shaker incubator at 37° C. while shaking. Then 3 ml of sample was taken therefrom and 3 ml of a fresh PBS was added back. Then O.D. was measured from the obtained sample using a spectrophotometer (UV-1800, Shimadzu, Japan), from which the concentration of the drug released was determined at each of the indicated days in FIG. 2 to calculate the cumulative amount of the drug released as shown in FIG. 2.

FIG. 2 shows the cumulative amount of drug released from the coated prostheses. As shown in FIG. 2 the drug was released for 5 days from the group TR_IM which received the prosthesis coated only with the drug. In contrast, PLGA_TR_IM group which received the prosthesis coated with the formulation comprising the polymer and the drug at the ratio of 100:1 released drug for 14 days. This indicates that the present formulation is effective in release controlling the drug and the sustained drug release can be extended above 15 days when the amount of the polymer contained in the formulation is 100 times more than the drug. As a result, the suppression of the scar formation was more prominent in the group where the drug was released over a longer period time. These results are agreed with the collagen density measured in the fibrosis tissue (scar tissue).

### Example 3

### Sutures Tested on Mice

### Establishment of Experimental Animals

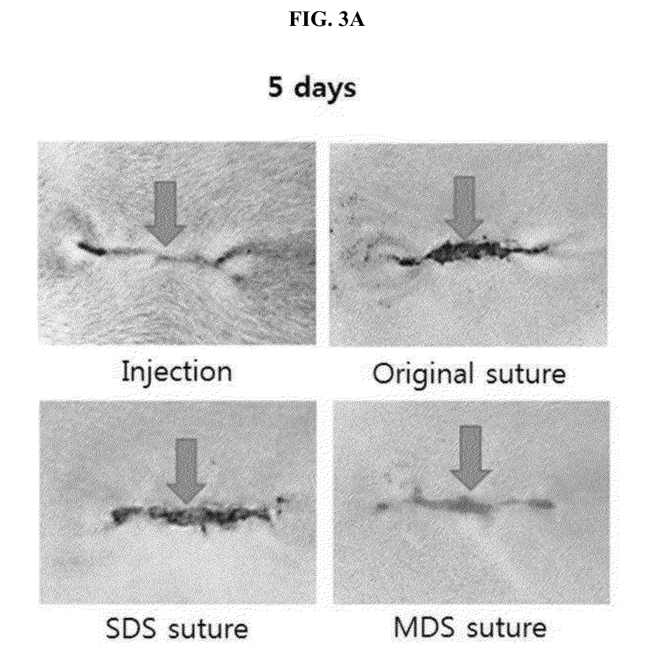

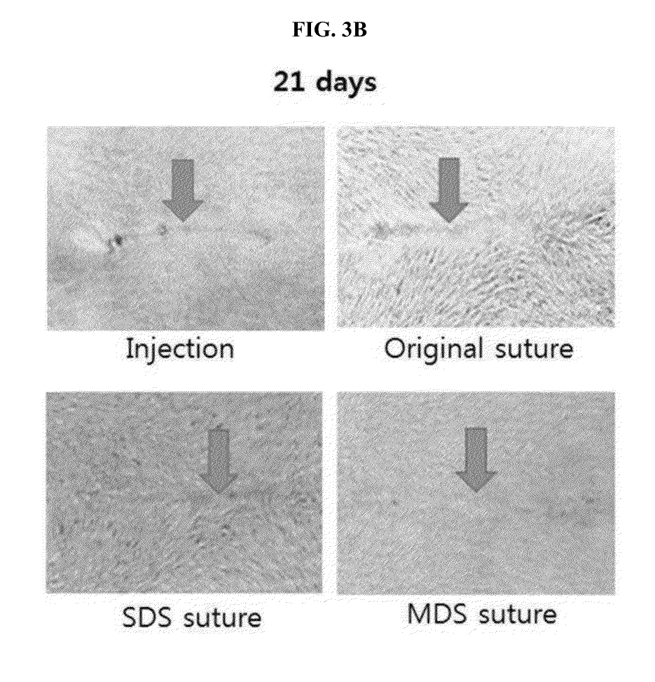

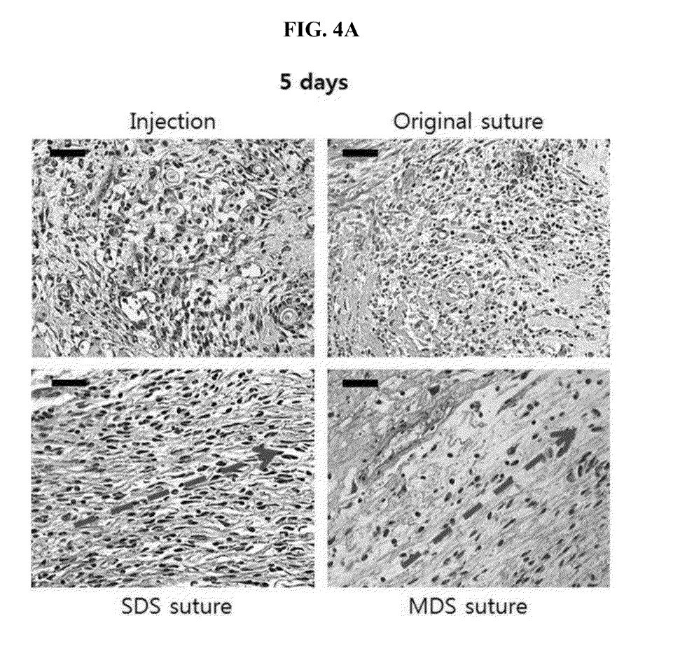

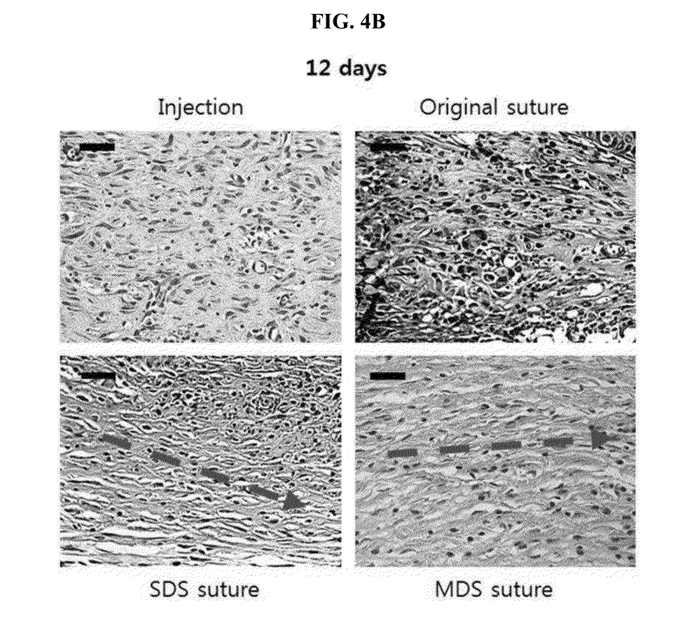

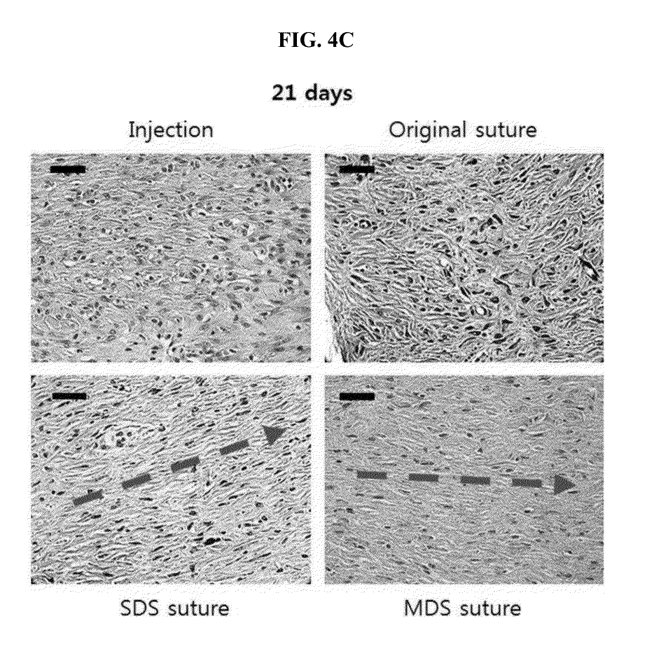

A mouse model was established to test the sutures coated with the present formulation as in Example 1. An oval shape incision of 3×1 cm in size was made on the back of each of five SD mice of 9 weeks old (Oriental Bio, Korea) to induce a wound. The wound was then left so that the tension was generated on the wound naturally. The tension is a main factor in forming the scar tissue and is able to induce scar formation. After inducing wound by an incision, the incision was closed inside of the incision using the present sutures and samples were taken from the wound to examine the appearance (FIGS. 3A and 3B) and directions of the collagen (FIGS. 4A to 4C) to determine the degree of the scar formation.

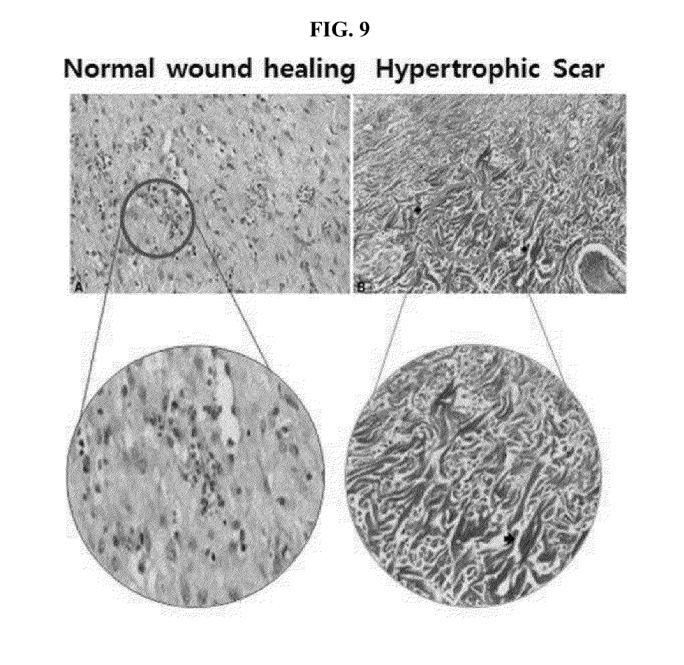

Scars are developed by abnormal reactions of cells during the tissue regeneration process at the wound site. Scars are composed of collagens generally generated during the wound healing process and are formed due to the difference in the accumulation of collagen. Scars are generally found in all the wounds. As shown in FIG. 9, in the scar tissue, no direction of the collagen was found in contrast to the normal healing process. These phenomena may be examined using a staining such as H&E staining. Particularly, hypertrophic wounds are characterized by the directionality of the collagen accumulated.

In FIGS. 3A and 3B, the injection group indicates the cases in which the incision was closed using a normal surgical suture and a solution type of bolus drug was given in the peripheral region of the wound to simulate the presence of drug for one day. The original suture group indicates the case in which the incision was closed using a normal surgical suture without a drug injection. The SDS suture and MDS suture groups indicate the cases in which each of the incision was closed using sutures coated with the coating formulation SDS (3 day drug release) and MDS (10 day drug release), respectively.

As shown in FIGS. 3A and 3B, at five days after the closure, there were nearly no differences observed among the groups because it was a too early stage to observe any healings. At 21 days after the closure, scars were found to be formed in Injection and Original suture groups. In contrast, in SDS (3 day drug release) and MDS (10 day drug release) groups in which the incisions were closed with sutures loaded with the drug using the present formulation, there were found no scars externally after 21 days, which is known as a period for the complete healing. These data indicate that the longer the period of the sustained drug release becomes, the more effectively the scar formation is suppressed. And at least 3 days of the sustained drug release is required for an effective suppression. In contrast, the group (Injection group) which was exposed to the drug only for one day was found to form scar tissues.

At 5, 12 and 21 days after the closure, tissue samples were obtained from the wound site. Then the paraffin sections were prepared from the sample and subject to a H&E (Hematoxylin and Eosin) staining to examine the directionality of the collagen. Results are shown in FIGS. 4A to 4C, no directionality was found in Injection and Original groups, which indicates the formation of scar tissue. In contrast, in SDS and MDS groups, the collagen was formed with directionality. This indicates a normal healing process undergoing at the wound site.

At 12 days after the closure, a second biopsy from the wound site was performed and directions of the collagen formed were examined. In consistent with the results from the 5 day samples, barely any directionality was found in Injection and Original groups. In contrast, in SDS and MDS groups, the collagen was formed with directionality. This indicates a normal healing process (thus no scar formation) undergoing at the wound site.

At 21 days which is considered a duration enough for a complete healing, a third biopsy from the wound site was performed and directions of the collagen formed were examined As results, no directionality was found in Injection and Original groups, which indicates the formation of scar tissue. In contrast, in SDS and MDS groups, the collagen was formed with directionality. This indicates a normal healing process undergoing at the wound site without the formation of excessive scar tissue due to the sustained release of the drug over the period of healing process.

### Example 4

### Prosthesis Implant Tested on Mice

### Establishment of Experimental Animals

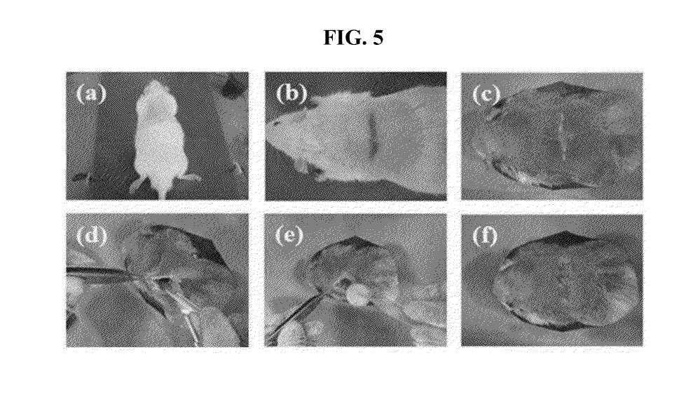

To perform the experiment using the prosthesis coated with the formulation as prepared in Example 1, a mouse model was established as shown in FIG. 5. An incision of 3 cm in length was made on the back of each of five SD mice of 9 weeks old (Oriental Bio, Korea). Then the prosthesis was inserted into the incision, after which the incision was closed.

Normally when the foreign materials such as prosthesis were implanted into the body, a reaction occurs to isolate the implanted prosthesis by synthesizing collagens at the peripheral regions of the implant. This results in the scar formation and the main component of the scar tissue is a collagen. Thus the isolation of the implanted prosthesis through a collagen forming reaction at the peripheral region leads to thick scar tissues, which is thus used as one of the factors to evaluate a degree of the side effects. Thus as described in Example 2, the prostheses as prepared in Example 2 were tested to determine whether the controlled release of the drug using the present formulation can suppress the formation of scar tissue caused by collagen synthesis and also can reduce the density of collagen at a wound site to suppress the formation of scar tissue.

To determine the thickness of scar tissues, tissues were obtained at 1, 2, 4, 8 and 12 after the implant from the peripheral regions of the implanted site and embedded into paraffin blocks which were then sectioned for H&E staining.

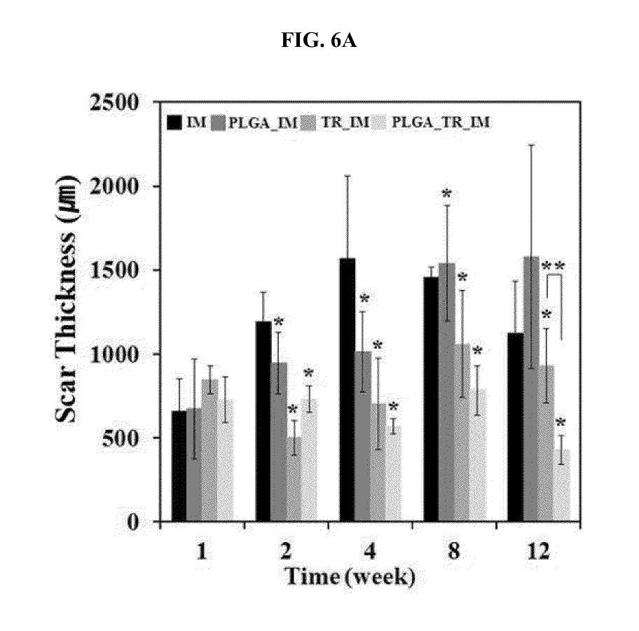

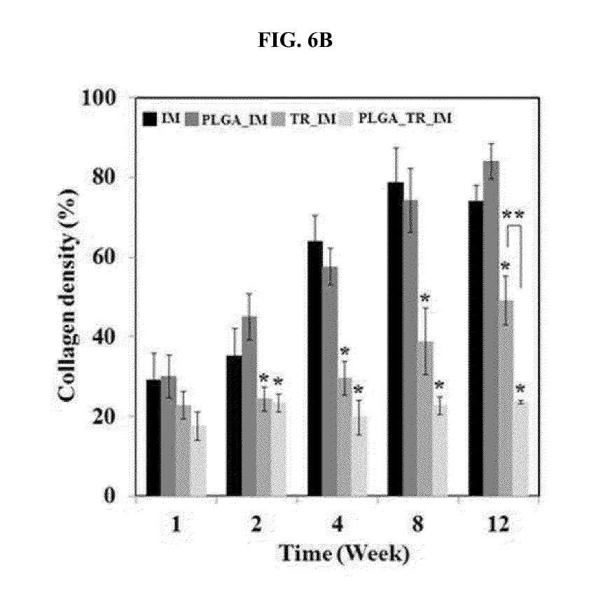

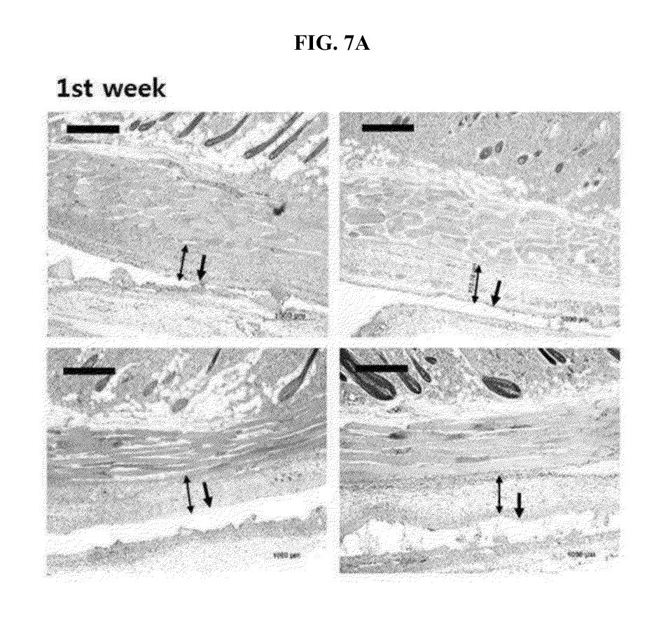

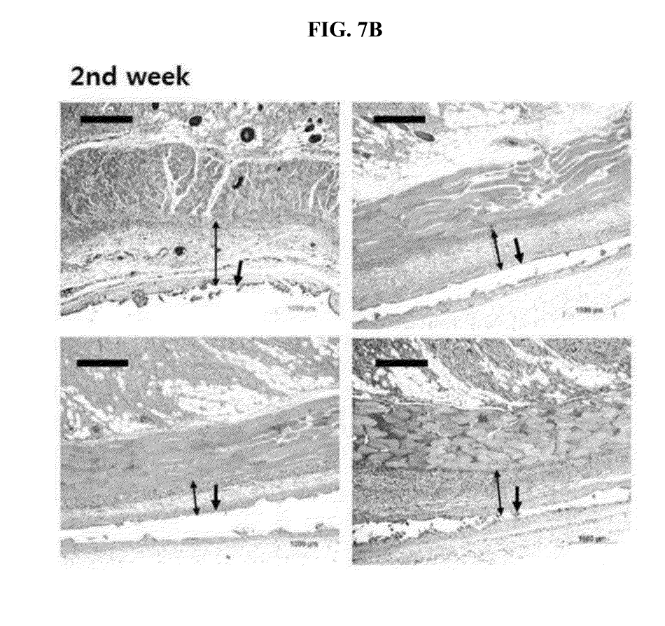

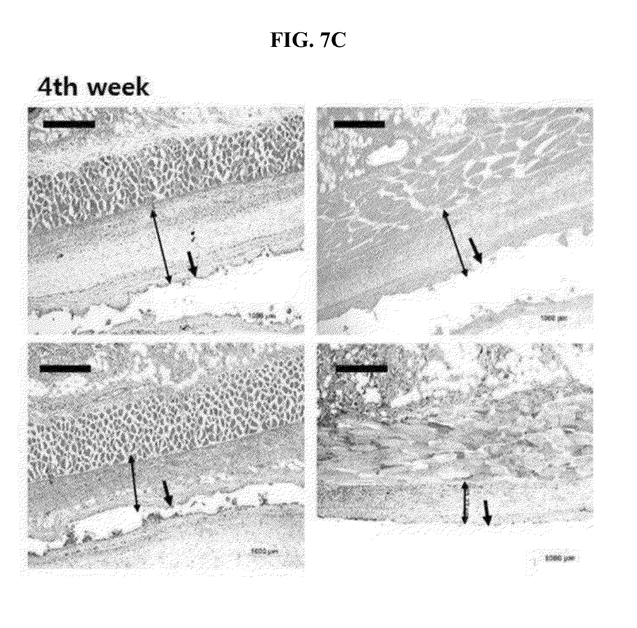

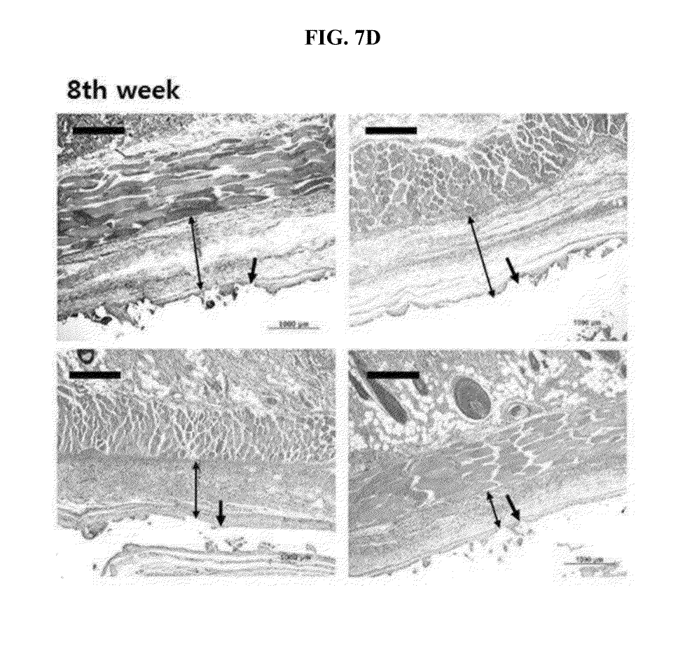

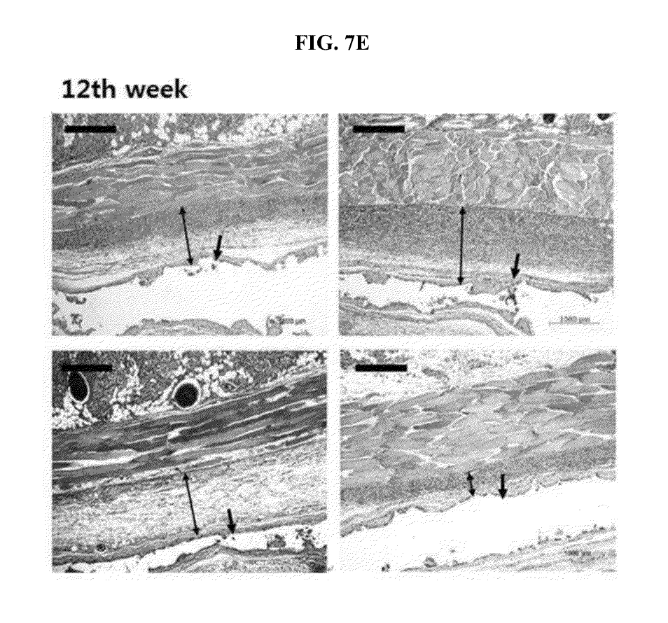

Results are shown in FIG. 6A and FIGS. 7A to 7E. The thickness of the scar was indicated as two arrows at both sides. The thicknesses were measured from at least 5 different sites from the scar forming region including a site that showed a least thickness. The values obtained then were averaged. Positions of the prosthesis are indicated as a black arrow.

As shown in FIGS. 6A and 7B, the differences in the thickness started to appear from 2 weeks after the implant and the groups implanted with the prosthesis coated with the present formulation were shown to develop a reduced scar formation. That is, two groups TR_IM which released the drug for 5 days and PLGA_TR_IM which released the drug for 14 days using the prosthesis coated with the present formulation resulted in a thickness which is thinner than that of IM group in which the prosthesis was not coated with the present formulation. It was also confirmed that the data were statistical significant.

Also as shown in FIGS. 6A and 7C, the differences in the thickness were observed even after 4 weeks between the experimental and control groups, and in the control groups the scar was continued to be formed thus thickness of the scar was increasing. In the experimental group TR_IM which released the drug over a period of 5 days, the thickness of the scar tissue was kept at a low level indicating the formation of scar tissues at a low level compared to PLGA_TR_IM group which released the drug over a period of 14 days. Overall, in the experimental groups, the formation of scar tissues was suppressed in compared to the control groups.

As shown in FIGS. 6A and 7E, at 12 weeks after the implant, it was found that the thickness of the scar was found to be thinner in the experimental groups TR_IM and PLGA_TR_IM, particularly in PLGA_TR_IM group which released the drug over a period of 14 days.

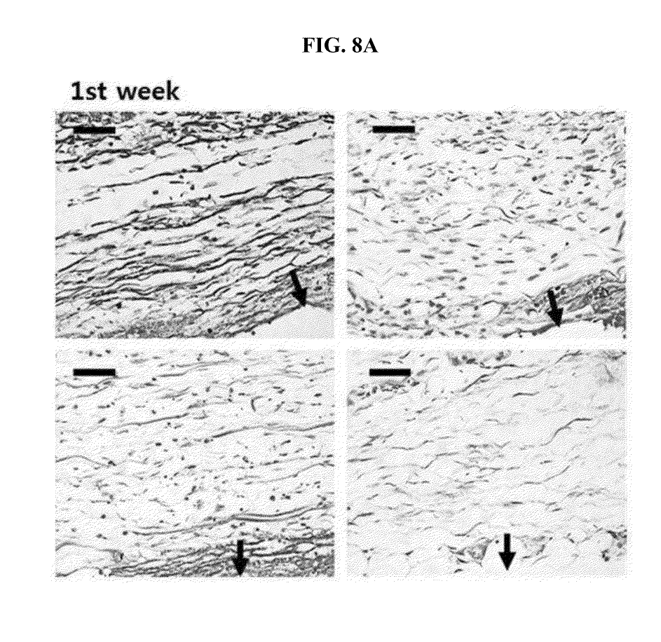

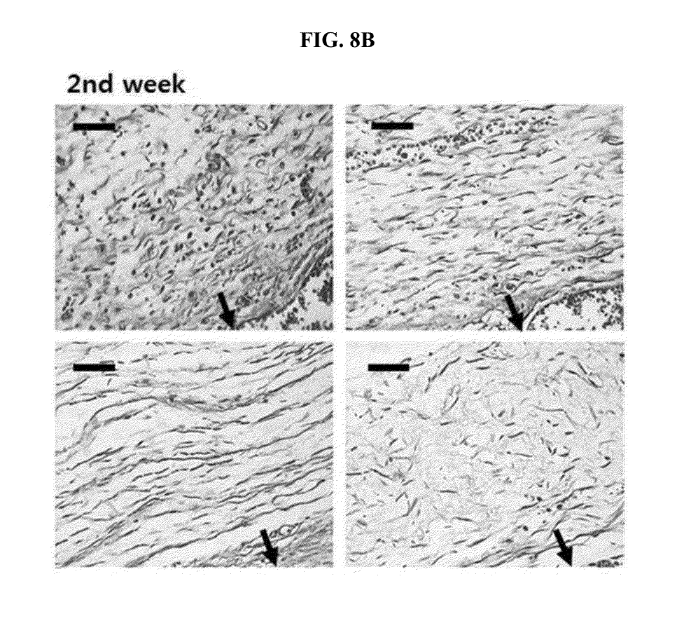

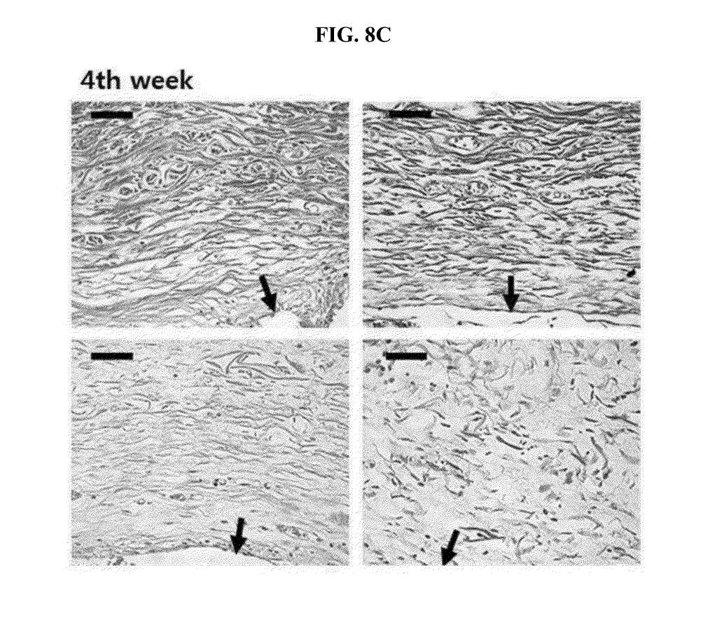

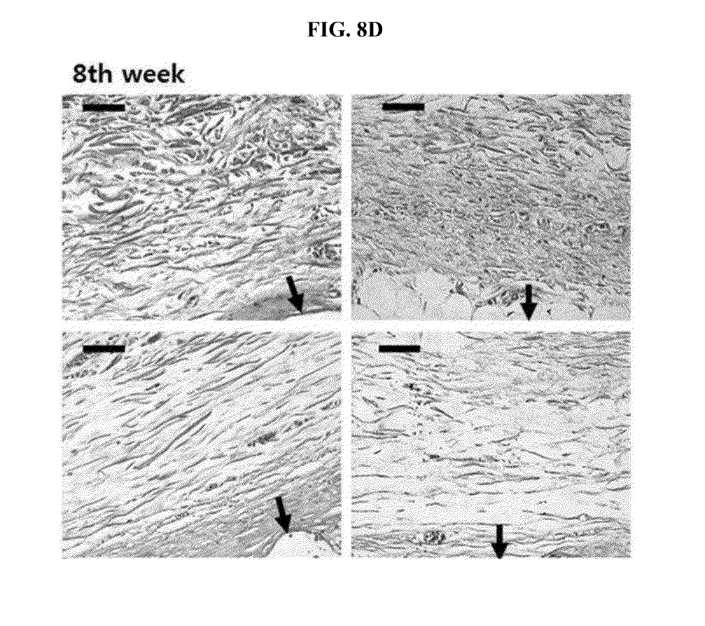

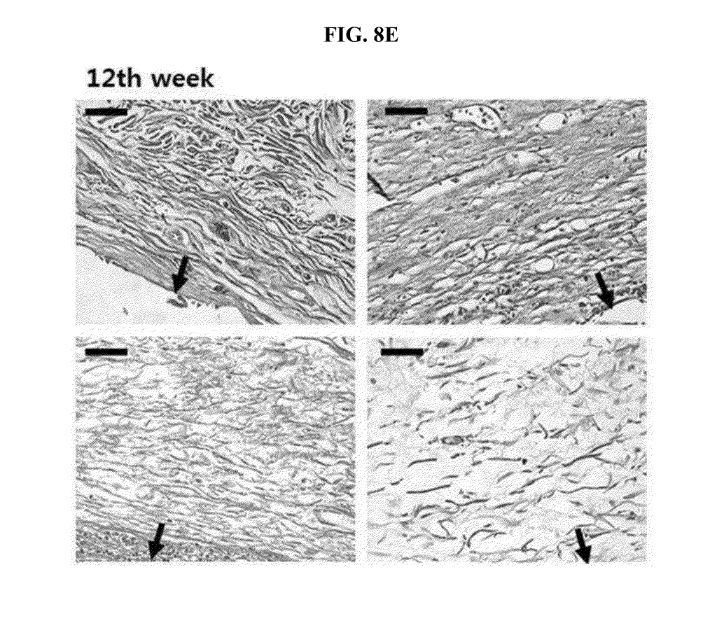

To determine the density of collagen, tissues were obtained at 1, 2, 4, 8 and 12 after the implant from the peripheral regions of the implanted site and embedded into paraffin blocks which were then sectioned for Masson's trichrome staining (Sigma Aldrich, USA) following the manufacturer's instruction by which the collagen is stained blue. The stained results were analyzed to determine the density using Image J program (Wayne rasband national institute of heath, USA). Results are shown in FIGS. 6A and 8A to 8E, in which the prostheses were indicated as black arrows.

As shown in FIGS. 6A and 8A, at 1 week, there was no statistically significant difference in the thickness found among the groups.

As shown in FIGS. 6A and 8B, at 2 weeks, the density of collagen was found higher in the control groups (IM and PLGA_IM) compared to the experimental groups (TR_IM and PLGA_TR_IM). And in comparison to IM group, statistically significant difference in the density of collagen was found in all the other groups. This indicates that the drug effectively suppressed the synthesis of collagen which plays a major role in the scar formation.

As shown in FIGS. 6A and 8C, at 4 weeks, it was found that the density of collagen continued to be increased in IM and PLGA_IM groups. In contrast the level was found below in the experimental groups (TR_IM and PLGA_TR_IM) indicating that the synthesis of collagen was effectively suppressed. And in comparison to IM group, statistically significant difference in the density of collagen was found in all the other groups. This indicates that the drug effectively suppressed the synthesis of collagen which plays a major role in the scar formation.

As shown in FIGS. 6A and 8D, at 8 weeks, it was found that the density of collagen steadily continued to be increased in IM and PLGA_IM groups. In contrast, in PLGA_TR_IM group, it was found that the collagen synthesis was suppressed due to the sustained release of the drug. In case of TR_IM group, the collagen synthesis was found to be increased at a low level. This indicates that in cases where the drug is released for 5 days, the effect from the drug becomes weaker after 4 weeks. Also, it was found that the drug was effective over 8 weeks in cases where the drug was released for 14 days. Overall the results indicate that the collagen synthesis thus the formation of scar tissue is able to be suppressed by the sustained and controlled release of drug using the present formulation.

As shown in FIGS. 6A and 8E, at 12 weeks, particularly in case of TR_IM group, the collagen synthesis was found to be increased and thus statistically significant difference in the density was found in comparison to PLGA_TR_IM group in which the drug was sustained released over a period of 14 days. This indicates that the drug release over a longer period of time (14 days) is more effective in suppressing the synthesis of collagen and thus the inhibition of scar formation, in which the drug has been effective for at least 12 weeks.

The various singular/plural permutations may be expressly set forth herein for sake of clarity. Although a few embodiments of the present disclosure have been shown and described, it would be appreciated by those skilled in the art that changes may be made in this embodiment without departing from the principles and sprit of the invention, the scope of which is defined in the claims and their equivalents.

What is claimed is:

1. A formulation for coating a medical device comprising an anti-scarring agent and a biocompatible polymer for controlling the release of the anti-scarring agent, wherein the ratio of the biocompatible polymer to the anti-scarring agent is from 100:15 to 100:0 by weight in which 0 is not included.

2. The formulation of claim 1, wherein the agent is one or more selected from the group consisting of acetmetacin, acrivastine, aldosterone, antazoline, astemizole, azatadine, azelastine, beclometasone, betamethasone, bromfenac, buclizine, carprofen, cetirizine, chloropyriline, chloropheniramine, clemastine, cromolyn, cyclizine, cyproheptadine, dexamethasone, diazoline, diclofenac, diphenhydramine, ebastine, emedastine, epinastine, etodolac, fenbufen, fenoprofen, fexofenadine, fludrocortisone, flurbiprofen, flurometalone, hydroxyzine, ibuprofen, indometacin, ketoprofen, ketorolac tromethamine, ketotifen, levocabastine, levoceterizine, lodoxamide, loratadine, loteprednol, loxoprofen, medrysone, mepivacaine, mequitazine, methdilazine, methapyrilene, monteleukast, nabumetone, naphazoline, naproxen, nedocromil, norastemizole, norebastine, olopatadine, phenidamine, phenylephrine, oxatamide, oxymetazoline, pemirolast, pheniramine, picumast, prednisilone, promethazine, rimexalone, repirinast, sulindac, suprofen, zafirlukast, tetrahydozoline, terfenadine, tiaprofenic acid, tometim, tranilast, triamcinolone, trimeprazine and triprolidine.

3. The formulation of claim 1, wherein the biocompatible polymer is at least one selected from the group consisting of polylactide (PLA), polyglycolide (PGA), poly(lactic-co-glycolic acid) (PLGA), polyorthoester, polyanhydride, polyamino acid, polyhydroxybutyric acid, polycaprolactone, polyalkylcarbonate, ethyl cellulose, chitosan, starch, guargum, gelatin and collagen.

4. The formulation of claim 1, wherein the ratio of the biocompatible polymer to the anti-scaring agent is from 100:15 to 100:1.

5. The formulation of claim 1, wherein the biocompatible polymer is PLGA.

6. The formulation of claim 1, wherein the medical device is wound dressings, products for wound closure, disposable bands, medical sponges, artificial blood vessels, structures for treating urinary incontinence, structures for fixing organs, meshes for preventing stenosis, maxillofacial meshes, hernia meshes, silicone implants, fibrous structures for heart valves, sutures, purification filters for white blood cells, purification filters for blood, filters for intravenous injection, filters for blood transfusion, filters for dialysis, filters for heart lung machine, dental textiles, fibrous structures for cartilage regeneration, artificial ligament, or artificial kidneys.

7. The formulation of claim 1, wherein the anti-scarring agent is released over a period of at least 3 days.

8. The formulation of claim 1, wherein the release of the anti-scarring agent is controlled by at least two properties of the biocompatible polymer including a hydrophobicity, a molecular weight, a structure of network and a degradation rate.
